(19) United States
(12) Patent Application Publication
(10) Pub. No.: US 2015/0065808 A1
(43) Pub. Date: Mar. 5, 2015

(54) SIMPLIFIED ARTHROSCOPY CANNULA

(71) Applicant: Hanshi LLC, Brazoria, TX (US)

(72) Inventors: Robert A. VAN WYK, St. Pete Beach, FL (US); Gary R. HEISLER, Brazoria, TX (US)

(73) Assignee: Hanshi LLC, Brazoria, TX (US)

(21) Appl. No.: 14/470,422

(22) Filed: Aug. 27, 2014

Related U.S. Application Data
(60) Provisional application No. 61/959,557, filed on Aug. 27, 2013.

(51) Int. Cl. A61B 17/34; A61B 17/02

(52) U.S. Cl. CPC A61B 17/3423; A61B 17/0218
USPC 600/208; 600/201

(57) ABSTRACT

An arthroscopic sealing cannula having improved efficiency, access and reduced manufacturing costs is described herein. In particular, the present invention describes arthroscopic sealing cannulae in which the conventional thermal and chemical bonding means are eliminated and replaced with a mechanical joining system that utilizes mating fastener pairs integrally molded into the distal and proximal elements of a cannula so as to thereby provide a strong reliable joining of the elements. Such a mechanical system eliminates the need for costly capital equipment and specializing tooling as well as the material and environmental handling problems associated with conventional bonding techniques. Furthermore, in that the join may be readily confirmed through simple visual examination, the present invention also eliminates the need for complex, costly, and time-consuming validation procedures mandated by regulations in place to ensure proper integrity, strength, and reliability of the bond. Accordingly, arthroscopic sealing cannulae constructed in accordance with the principles of this invention are expected to have increased reliability and reduced manufacturing costs.

### PRIORITY

This application claims the benefit of U.S. Provisional Application No. 61/959,557 filed Aug. 27, 2013. The entire content of this priority application is incorporated by reference herein.

### FIELD OF THE INVENTION

The present invention relates to an efficient and simplified cannula for endoscopic surgery.

### BACKGROUND OF THE INVENTION

Arthroscopic procedures generally involve the passage of elongated instruments through portals that facilitate access to the internally located surgery site. Because these sites are generally filled with liquid under pressure, the use of a sealing access device is required. It is required that this access device, commonly called a sealing cannula or simply a cannula, provide for easy insertion, manipulation and retraction of instruments, and while also maintaining a fluid seal to prevent uncontrolled escape of pressurized fluid from the site. This sealing must be maintained both when instruments are in use as well as when there are no instruments within the cannula passageway. Commercially available examples of such arthroscopy cannulae include the Clear-Trac cannulae by Smith and Nephew (Andover, Mass.), the Dri-Loc Disposable Cannulas by Stryker, Inc. (Kalamazoo, Mich.), and the Twist-In Cannulas by Arthrex, Inc. (Naples, Fla.).

A typical arthroscopy cannula has three principle elements: an elongate tubular distal element which is positioned within an incision made in the skin of a patient, one or more elastomeric sealing elements which prevents escape of fluid from the fluid-filled joint space when elongate instruments are inserted into the cannula, and a proximal portion which retains the seal in its position in the fluid/instrument path. Typically, the one or more sealing elements are positioned in a cavity formed between the distal and proximal elements, and the distal and proximal elements are joined by ultrasonic welding, solvent bonding, or use of a bonding agent such as, for instance, epoxy, cyanoacrylate or other curable adhesive. The sealing elements are typically formed from an elastomeric material such as silicone. The distal and proximal elements are typically made from a rigid polymeric material, although in some cases the distal element is formed from a non-rigid polymeric material to allow the passage of irregularly shaped instruments.

Joining of the distal and proximal elements by ultrasonic welding or solvent bonding is problematic in that the integrity of the bond is difficult to confirm. Regulatory agencies require that the joining process be validated, that is, through testing and statistical analysis demonstrating that the bond formed meets strength and reliability specifications. However, even when the joining process is validated, variations within the process may occur that weaken the bond to the point where failure may occur during use. Such variations that lead to failure are not detectable, and unless statistically designed on-going destructive testing of the finished product is performed during production, large numbers of product with weak bonds may be supplied to customers. The validation of the bonding process is a costly time-consuming procedure that gives only limited assurance of the bond integrity.

A second problem in the art of arthroscopic cannulae arises with the use of irregularly shaped instruments, the passage of which can cause deformation of the sealing elements, thereby allowing pressurized fluid from the site to escape. This may also occur when sutures extending from the site through the cannula and exiting from the cannula's proximal end are placed under tension, as when tying knots. The escaping liquid frequently comes out as a stream directly at the surgeon who is passing the instruments or tensioning the suture. Because of this, some manufacturers have begun adding an auxiliary sealing means to the proximal end of the cannula to prevent leakage. One example of such a device is disclosed in U.S. Pat. No. 5,779,697 to Glowa et al. The Glowa device includes an elastomeric sealing member mounted to the proximal end of the cannula in addition to a more distally mounted elastomeric seal so as to prevent leakage when instruments are inserted, retracted or mis-aligned. This same approach is used in Instrument Cannulas by Arthrex, Inc. (Naples, Fla.) that are supplied to surgeons with a “no squirt” elastomeric member attached to the cannula's proximal end. An alternative approach to dealing with leakage due to deformation of the sealing element is taught by Morris et al in U.S. Pat. No. 7,993,355 wherein a suture organizing device is provided with an elastomeric “spray shield” that is removably mounted to the proximal end of a cannula, the spray shield being configured not to prevent leakage, but rather to deflect the flow of escaping pressurized fluid using deformable flaps formed in the element. Escaping liquid does not spray at the surgeon, but rather flows from the device as a low-velocity stream. The liquid may exit the device by deforming the flaps, or alternatively, through holes in the spray shield at the proximal end of the flap-forming slots. In either case, pressurized liquid escaping past the seal at high velocity exits the device as a low-velocity stream. Dooney et al in U.S. Patent Publication 2014/0121630 teaches the same spray shield approach but with the spray shield integral to the cannula. In particular, Dooney teaches “ . . . an adjacent outer “baffle-like dam” that prevents fluid pressure build-up and allows the fluid to leak out and not squirt out of the cannula”. The “baffle-like” dam has slots that form flaps, and holes for the escape of fluid in the same manner as Morris. While the constructions of the Dooney device is simple, Dooney teaches Cap 65 may be attached by any known method in the art, for example, by welding such as ultrasonic welding.” Known methods would include solvent bonding and adhesive bonding in addition to ultrasonic welding. However, the drawbacks of these joining methods have been previously herein described.

In contemplating means to address the aforementioned problems, the skilled artisan must keep in mind that not all arthroscopic instruments are straight. Various devices such as shaver blades are curved, yet are advantageously brought to the surgical site via a cannula, Also, some devices, particularly some manual instruments, have irregular shaped distal portions which will not fit into a standard round cannula. To accommodate these devices, sealing cannulae having a flexible polymeric distal portion have been developed. The distal portions of these cannula will bend to accommodate curved devices placed within them, or their lumen will deform to allow the passage of devices which would not fit through a conventional circular cross-sectioned lumen. Commercial examples of such alternative sealing cannulae include the Clear-Trac Flexible Cannula System by Smith and Nephew, Inc. (Andover, Mass.), and the Hex-Flex Cannulas by Conmed, Inc. (Largo, Fla.). These cannulae have construction similar to that of rigid cannulae in that they require bonding between structural elements and may limit the degree of flexibility which may be imparted to the distal portion. This, in turn, limits the functionality of the cannula since a flexible cannula with a high degree of rigidity (resistance to deformation) will make passage of irregularly shaped or bent device difficult.

Accordingly, there is a need in the art for a cannula that may be manufactured without ultrasonic welding, and without adhesive or solvent bonding. There is further a need for a cannula that incorporates an elastomeric spray shield and may also be manufactured without ultrasonic welding and without adhesive or solvent bonding. Finally there is also a need for a cannula with a flexible distal portion in which the properties of the distal portion are not limited by the assembly bonding process.

### SUMMARY OF THE INVENTION

In the course of researching the afore-mentioned problems in the arthroscopic arts, the present inventors discovered one could eliminate the need for a bond between the distal and proximal elements of a cannula through the use of a suitable mechanical joining means provided in the configuration of the elements. Specifically, one could configure the elements such that mating fastener pairs are integrally molded into the distal and proximal elements of a cannula so as to thereby provide a strong reliable joining of the elements. The finished devices may be visually inspected to ensure that the fastener pairs are properly engaged so as to ensure the integrity of the joining means. Assembly of a cannula constructed in accordance with the principles of this invention may be rapidly accomplished without requiring capital equipment and specialized tooling as in the case with ultrasonic welding of the components, and without the environmental and material handling problems inherent in solvent bonding. Accordingly, cannulae constructed in accordance with the principles of this invention will have increased reliability and reduced manufacturing costs.

In accordance with the present invention, these same construction techniques—using integral fastener pairs on the proximal and distal elements—may be advantageously applied to cannulae that have a proximally positioned elastomeric spray shield integral to their assembly, and may also be applied to cannulae that have flexible distal assemblies, wherein the mechanical properties of the distal portion are not limited by the manufacturing methods used.

Accordingly, it is an objective of the present invention to provide a cannula assembly comprising:
a. a proximal hub element having (i) a central opening configured to receive surgical instruments, (ii) a planar annular body portion that includes a first component of a mating fastener pair, and (iii) a distally projecting flange portion;
b. a distal tubular element composed of (i) an elongate tubular distal portion, (ii) a flared proximal portion that includes a second component of the mating fastener pair, and (iii) a proximally facing raised rim extending from the flared proximal portion, and
c. one or more sealing membranes,
   wherein components (a)-(c) are assembled together such that the first and second components of the mating fastener pair mechanically interlock so as to securely fasten the proximal hub element to the distal tubular element and prevent relative movement and/or disengagement thereof.

It is a further object of the present invention to provide novel spray shield assemblies for use with the cannulae of the instant invention and/or conventional arthroscopic cannula.

It is yet a further object of the present invention to provide a proximal hub element and distal tubular element that are each integrally molded from a rigid polymeric material. Alternatively, the distal tubular element, particularly elongate tubular distal portion may be composed of a flexible, elastomeric material and designed to accommodate curved and irregularly shape instruments. The novel fastening and spray shield systems disclosed herein may be accommodated to fit either configuration.

In a preferred embodiment, the fastener pair is composed of integral projecting hooks that mate with corresponding integral recessed elements. In a particularly preferred embodiment, the hooks and the recessed elements feature coordinating beveled portions or projections. Depending on the construction of the respective mating components of the fastener pair, the bond between the proximal and distal elements of the assembly may be permanent (i.e., as in a single use device). To that end, the present invention contemplates simple mechanical fits as well as thermal techniques such as heat staking to ensure irremovable engagement. Alternatively, the cannula assembly of the present invention may be designed for repeated disassembly (i.e., as in a multi-use device) and reassembly, for example with replacement sealing membranes or the like.

These and other objectives are accomplished in the invention herein described, directed to a simplified, more efficient, low cost arthroscopy cannula. Further objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention.

### BRIEF DESCRIPTION OF THE DRAWINGS

Various aspects and applications of the present invention will become apparent to the skilled artisan upon consideration of the brief description of figures and the detailed description of the present invention and its preferred embodiments that follows:

FIG. 1A depicts an exploded proximal perspective view of a prior art arthroscopic sealing cannula.

FIG. 1B is a plan view of a prior art arthroscopic sealing cannula.

FIG. 1C is a perspective view of the prior art arthroscopic sealing cannula of 1B.

FIG. 2A depicts an exploded proximal perspective view of a novel arthroscopic sealing cannula formed in accordance with the principles of this invention.

FIG. 2B is an exploded distal perspective view of the objects of FIG. 2A.

FIG. 3A is a plan view of the distal element of the cannula of FIGS. 2A and 2B.

FIG. 3B is a side elevational sectional view of the objects of FIG. 3A at location A-A of FIG. 3A.

FIG. 3C is an expanded view of region A of FIG. 3B.

FIG. 4A is a plan view of the proximal element of the cannula of FIGS. 2A and 2B.

FIG. 4B is a side elevational sectional view of the objects of FIG. 4A at location A-A of FIG. 4A.

FIG. 4C is an expanded view of region A of FIG. 4B.

FIG. 5A is a perspective view of a cannula formed in accordance with the principles of this invention.

FIG. 5B is an expanded proximal end view of the objects of FIG. 5A.

FIG. 6A is a plan view of the cannula of FIG. 5A.

FIG. 6B is an expanded side elevational sectional view of the proximal portion of the objects of FIG. 6A at location A-A of FIG. 6A.

FIG. 6C is an expanded view of region A of FIG. 6B

FIG. 7 is a side elevational view of the objects of FIG. 6A.

FIG. 8 is an expanded plan sectional view of the distal portion of the objects of FIG. 7 at location B-B of FIG. 7.

FIG. 9A is a plan view of a first alternate embodiment cannula formed in accordance with the principles of the instant invention.

FIG. 9B is a side elevational sectional view of the objects of FIG. 9A at location A-A.

FIG. 9C is an expanded view of region A of FIG. 9B.

FIG. 10A is an exploded perspective assembly view of the alternate embodiment cannula of FIG. 9.

FIG. 10B is a perspective view of the alternate embodiment cannula of FIG. 9.

FIG. 11 is an exploded perspective assembly view of the components of a second alternate embodiment cannula formed in accordance with the principles of this invention prior to final assembly.

FIG. 12A is a distal perspective view of the objects of FIG. 11 assembled in preparation for heat staking.

FIG. 12B is a side elevational view of the objects of FIG. 12A.

FIG. 13A is a distal perspective view of the objects of FIG. 12A after completion of assembly by heat staking.

FIG. 13B is a side elevational view of the objects of FIG. 13A.

FIG. 13C is a proximal perspective view of the objects of FIG. 13A.

FIG. 14 is a proximal perspective view of the tubular body element of a spray suppression assembly for assembly to a cannula constructed in accordance with the principles of this invention.

FIG. 15 is a distal perspective view of the objects of FIG. 14.

FIG. 16 is a distal axial view of the objects of FIG. 14.

FIG. 17 is a plan view of the objects of FIG. 14.

FIG. 18 is a proximal axial view of the objects of FIG. 14.

FIG. 19 is a side elevational sectional view of the objects of FIG. 14 at location A-A of FIG. 17.

FIG. 20 is a side elevational view of a flexible polymeric spray shield for a spray suppression assembly for assembly to a cannula constructed in accordance with the principles of this invention.

FIG. 21 is an axial view of the objects of FIG. 20.

FIG. 22 is a perspective view of the objects of FIG. 20.

FIG. 23 is a side elevational view of a retaining ring for a spray suppression assembly for assembly to a cannula constructed in accordance with the principles of this invention.

FIG. 24 is a side elevational view of the objects of FIG. 23.

FIG. 25 is a perspective view of the objects of FIG. 23.

FIG. 26 is a perspective view of the exploded assembly of a spray suppression assembly for mounting to a cannula constructed in accordance with the principles of this invention.

FIG. 27 is a proximal perspective view of the elements of FIG. 26 assembled to form a spray suppression assembly for mounting to a cannula constructed in accordance with the principles of this invention.

FIG. 28 is a distal perspective view of the elements of FIG. 27.

FIG. 29 is a distal axial view of the shaver suppression assembly of FIG. 27.

FIG. 30 is a side elevational view of the objects of FIG. 27.

FIG. 31 is a proximal axial view of the objects of FIG. 27.

FIG. 32 is a plan sectional view of the elements of FIG. 27 at location A-A of FIG. 30.

FIG. 33 is a proximal perspective depiction of the cannula of FIGS. 1 through 8 and the spray suppression assembly of FIGS. 24 through 32 positioned for assembly of the spray suppression assembly to the cannula.

FIG. 34 is a distal perspective depiction of the objects of FIG. 33.

FIG. 35 is a proximal perspective depiction of a cannula assembly composed of the cannula of FIGS. 1 through 8 and the spray suppression assembly of FIGS. 24 through 32.

FIG. 36 is a distal perspective view of the objects of FIG. 35.

FIG. 37 is a plan view of the cannula assembly of FIG. 35.

FIG. 38 is a side elevational sectional view of the objects of FIG. 35 at location A-A of FIG. 37.

FIG. 39 is a plan view of a flexible polymeric distal portion for a cannula constructed in accordance with the principles of the instant invention.

FIG. 40 is a side elevational view of the objects of FIG. 39.

FIG. 41 is an expanded axial sectional view of the objects of FIG. 39 at location B-B of FIG. 40.

FIG. 42 is an expanded side elevational sectional view of the objects of FIG. 39 at location A-A of FIG. 39.

FIG. 43 is a plan view of a proximal subassembly for an alternate embodiment cannula formed in accordance with the principles of this invention.

FIG. 44 is a perspective view of the subassembly of FIG. 43.

FIG. 45 is a side elevational view of the objects of FIG. 43.

FIG. 46 is an expanded side elevational view of the objects of FIG. 43 at location A-A of FIG. 43.

FIG. 47 is a plan view of a retaining collar for an alternate embodiment cannula formed in accordance with principles of the instant invention.

FIG. 48 is an axial view of the objects of FIG. 47.

FIG. 49 is a side elevational sectional view of the collar of FIG. 47 at location A-A of FIG. 47.

FIG. 50 is a perspective view of the objects of FIG. 47.

FIG. 51 is a proximal perspective view of an alternate embodiment cannula having a flexible distal portion and formed in accordance with the principles of the instant invention.

FIG. 52 is a distal perspective view of the cannula of FIG. 51.

FIG. 53 is a plan view of the objects of FIG. 51.

FIG. 54 is an expanded side elevational sectional view of the objects of FIG. 53 at location A-A of FIG. 53.

FIG. 55 is an expanded axial sectional view of the objects of FIG. 53 at location B-B of FIG. 53.

### DESCRIPTION OF THE PREFERRED EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims. Accordingly, unless otherwise defined, all technical and scientific terms used herein have the same meaning as commonly understood by one of ordinary skill in the art to which the present invention belongs. However, in case of conflict, the present specification, including definitions below, will control.

In the context of the present invention, the following definitions apply:

The words “a”, “an” and “the” as used herein mean “at least one” unless otherwise specifically indicated. Thus, for example, reference to an “opening” is a reference to one or more openings and equivalents thereof known to those skilled in the art, and so forth.

The term “proximal” as used herein refers to that end or portion which is situated closest to the user of the device, farthest away from the target surgical site. In the context of the present invention, the proximal end of the arthroscopic sealing cannula includes the hub region.

The term “distal” as used herein refers to that end or portion situated farthest away from the user of the device, closest to the target surgical site. In the context of the present invention, the distal end of the arthroscopic sealing cannula includes the elongate lumened region that passes through the incision site.

In the context of the present invention, the term “cannula” is used interchangeably to refer to the family of elongate surgical instruments that facilitate access across tissue to an internally located surgery site.

The terms “tube” and “tubular” are used herein to a generally round, long, hollow component having at least one central opening often referred to as a “lumen”.

In the context of the present invention, the terms “seal”, “sealing element” and “membrane” are used interchangeably to refer to any of the various shaped pieces or discs of rubber or other elastomeric material sealing the junction between two surfaces, particularly between the proximal and distal ends of an arthroscopic cannula of the present invention, or between an instrument placed in the lumen of the cannula and the cannula assembly so as to prevent liquid flow through the cannula.

The terms “lengthwise” and “axial” as used interchangeably herein to refer to the direction relating to or parallel with the longitudinal axis of a device. The term “transverse” as used herein refers to the direction lying or extending across or perpendicular to the longitudinal axis of a device.

The term “lateral” pertains to the side and, as used herein, refers to motion, movement, or materials that are situated at, proceeding from, or directed to a side of a device.

The term “medial” pertains to the middle, and as used herein, refers to motion, movement or materials that are situated in the middle, in particular situated near the median plane or the midline of the device or subset component thereof. In the context of the present invention, the terms “protrusion” and “protuberance” are used interchangeably herein to refer to a projecting element, such as a raised ridge, spline, or rib, that mates and/or engages with a coordinated recessed element, such as a groove or slot.

In the Examples below, the present invention makes reference to a mechanically fit and/or optionally heat-staked fastener pair that arises from the engagement of a distal hook element and a proximal recess element. However, the present invention contemplates the reversal of such elements, wherein the recesses are disposed on the distal tubular component and the hooks are disposed on the proximal hub element.

In the Examples below, the present invention also makes reference to various lock-and-key type alignment mechanisms that serve to establish and maintain proper angular alignment between the proximal hub element and the distal tubular element, as well as the optional spray shield assembly. It will again be readily understood by the skilled artisan that the position of the respective coordinating elements (e.g., mating slots and protrusions) may be exchanged and/or reversed as needed.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal.

Hereinafter, the present invention is described in more detail by reference to the Figures and Examples. However, the following materials, methods, figures, and examples only illustrate aspects of the invention and are in no way intended to limit the scope of the present invention. For example, while the present invention makes specific reference to arthroscopic procedures, it is readily apparent that the teachings of the present invention may be applied to other minimally invasive procedures and are not limited to arthroscopic uses alone. As such, methods and materials similar or equivalent to those described herein can be used in the practice or testing of the present invention.

### EXAMPLES

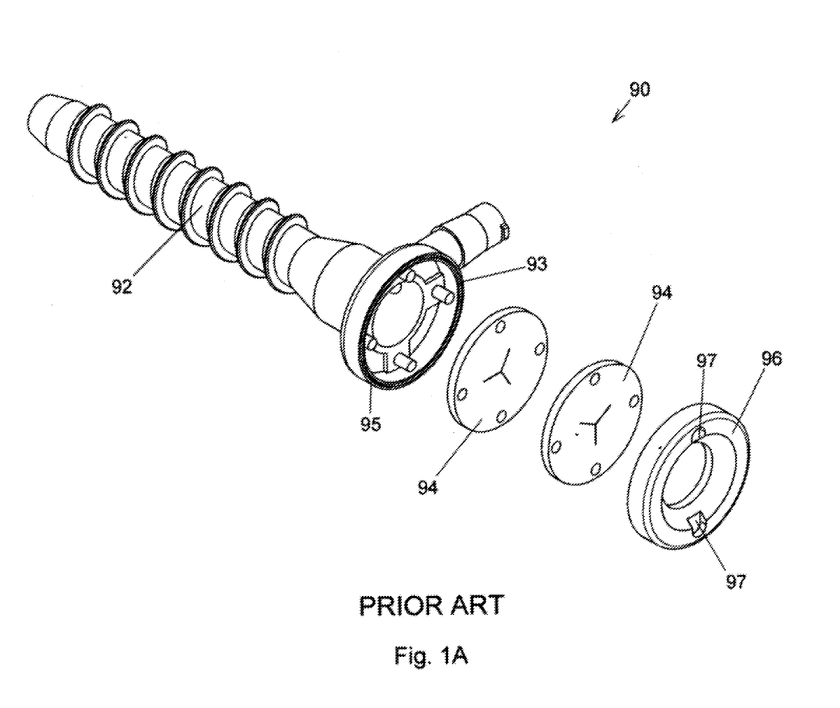

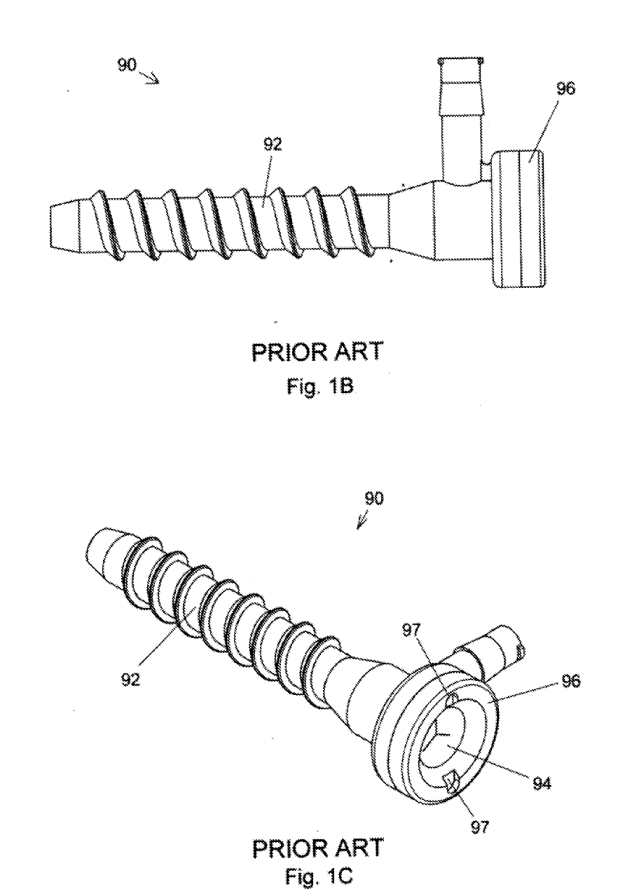

FIGS. 1A through 1C depict the construction of a typical prior art cannula, more particularly an arthroscopic sealing cannula. As best seen in FIG. 1A, prior art cannula 90 has a rigid polymeric distal element 92, one or more elastomeric membranes or seals 94, and a rigid polymeric proximal element 96 which is bonded to distal element 92 by ultrasonic welding, solvent bonding, or an adhesive, ultrasonic welding being the preferred method. Slots 97 in proximal element 96 allow cannula 90 to be inserted and retracted from a surgeon-formed portal in the body of a patient using a specialized handle called an obturator that allows the surgeon to apply axial force and torque to cannula 90 as needed. Cannula 90 as shown is configured for assembly by ultrasonic welding of distal portion 92 to proximal portion 96 with proximal facing annular surface 93 having formed thereon an annular ridge 95 which functions as an “energy director” to aid in forming the bond. When subjected to pressure and ultrasonic vibration, localized melting of ridge 95 provides material that flows between the proximal facing surface 93 and the distal facing surface of proximal portion 96. The size and configuration of annular ridge 95 is critical since if ridge 95 contains excess material the melted plastic may flow beyond the periphery of the joint, and if the material of the ridge 95 is deficient the bond may not have the specified strength. Deficiency in the material of ridge 95 (a common molding problem known as a “short shot”) may be due to changes in the parameters of the molding process used to form distal portion 92. Should such changes occur and be undetected prior to assembly of device 90 by ultrasonic welding, a cannula 90 with less than specified bond strength may be shipped to surgeons and fail during use, the failure mode being separation of proximal portion 96 from distal portion 92 when an instrument is retracted from the cannula. Such failure requires immediate attention, namely immediate replacement of the cannula, which, in turn, extends the procedure time, an undesirable outcome for both the surgeon and the patient. Verifying the integrity of the bond requires destructive testing of cannula 90 since visual inspection cannot detect substandard bonds. Because of this, molding and ultrasonic welding parameters must be closely controlled and periodic destructive testing of cannula 90 during the bonding process is required. Such testing increases the cost of production for cannula 90 since a portion of the products produced must be destroyed to verify bond integrity. Additionally, the ultrasonic welding machine with its associated tooling (commonly called a “horn”) for transmitting ultrasonic energy to the part must be validated according to procedures which conform to FDA regulations. That is, parameters must be established for the welding machine and molded components which produce bonds having a predetermined strength. Validating the process, machine and tooling requires destructive testing of large numbers of welded assemblies. If the tooling is changed or the machine undergoes maintenance or repairs that may affect the calibration of its output controls, the process must be requalified, again, a process that is time consuming and again requires the destructive testing of large numbers of welded assemblies.

Given the above-described issues associated with the status quo, replacing current bonding methods with mechanical fastening methods that may be visually inspected has significant benefits. In the course of researching alternatives, it was herein discovered one could produce a cannula in which interlocking features on distal and proximal elements of the cannula permanently and irretrievably affix the proximal portion to the distal portion in a manner which may be visually inspected. Accordingly, cannulae formed in accordance with the principles of the present invention do not use ultrasonic welding or bonding agents, but rather mechanical interlocking of features on the components to maintain the integrity of the final assembly.

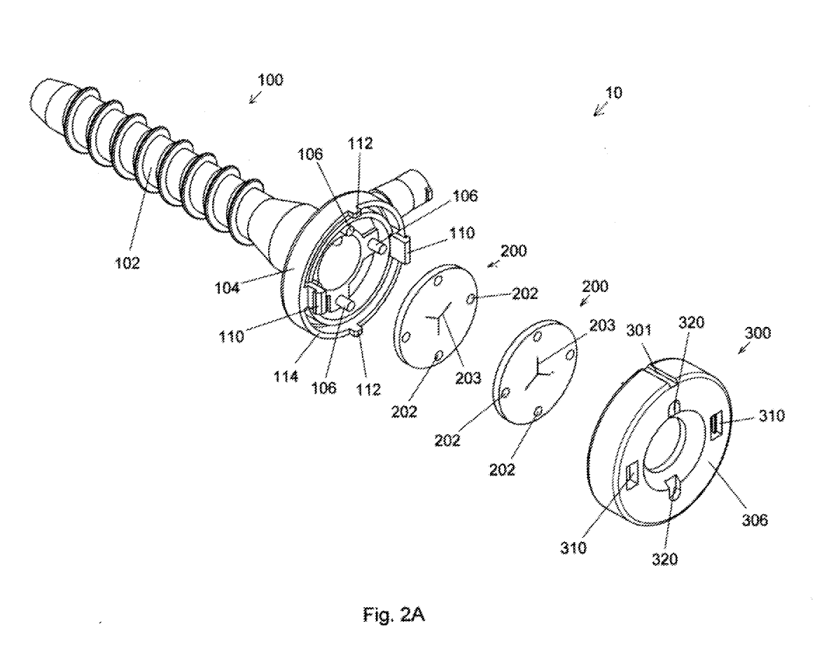

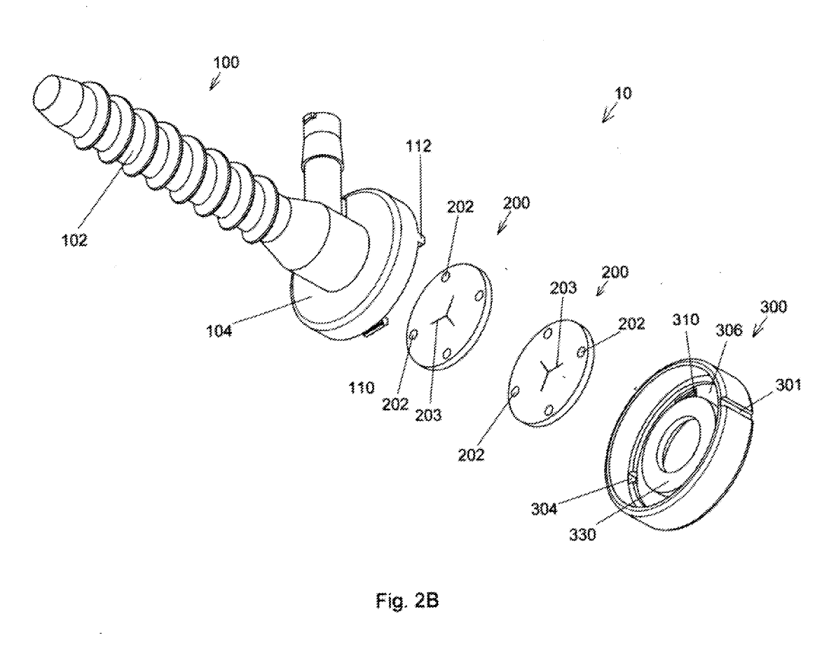

FIGS. 2A and 2B depict an exploded view of an arthroscopic cannula 10 constructed in accordance with the principles of this invention. Cannula 10 has an elongate tubular distal element 100, sealing elements 200, and a proximal hub element 300. Features of cannula 10 other than those related to mechanical joining of the distal and proximal elements, for example the external threads on the distal end, are like those of prior art devices and form no part of the present invention which is directed solely to the simple reliable joining of the respective elements.

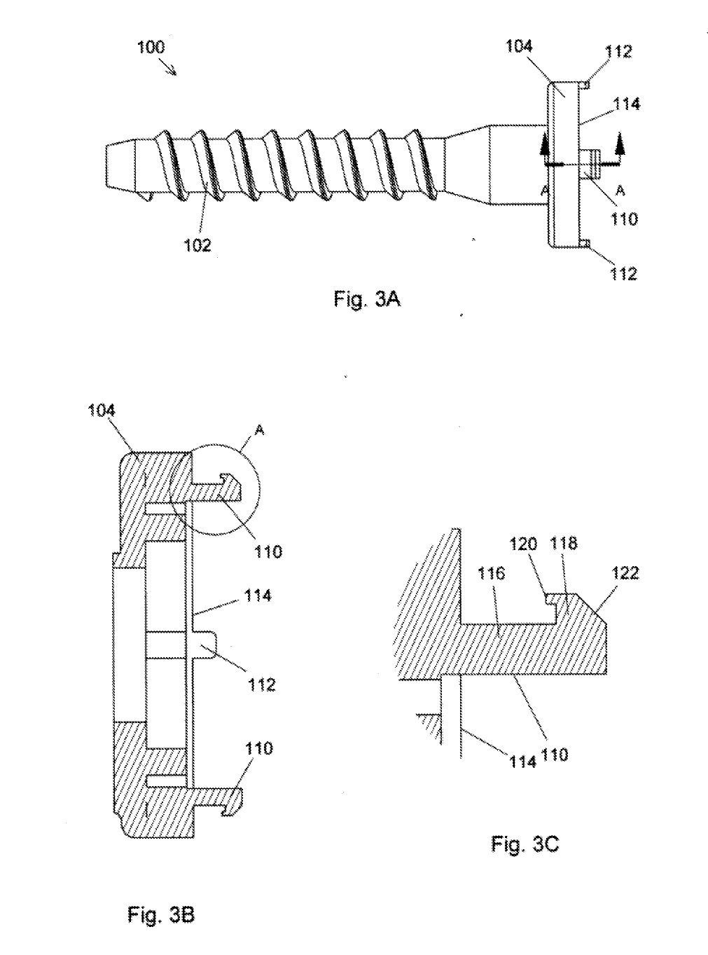

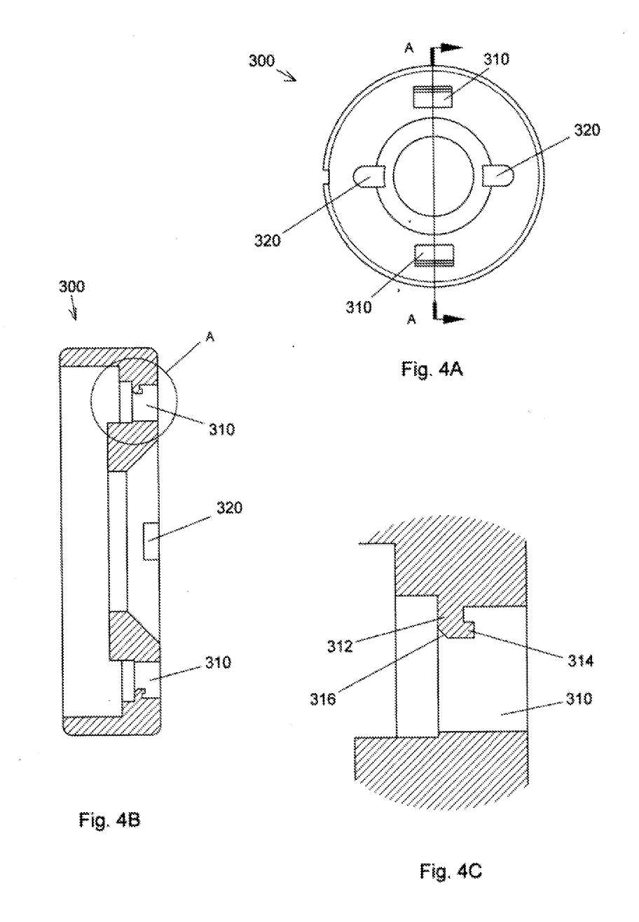

Distal element 100 has an elongate tubular distal portion 102 that may optionally be threaded. Proximal portion 104 locates and retains sealing elements 200 by means of pins 106 that engage with holes 202 in seal 200. Hook portions 110 protrude proximally from proximal portion 104. Proximal portion 104 also has alignment protrusions or splines 112 extending from proximal rim 114 of proximal portion 104 of distal element 100. Alignment protrusions 112 cooperatively engage with the slots 304 in flange element 306 of proximal element 300 to establish and maintain angular alignment between distal element 100 and proximal element 300. Recessed features 310 of proximal element 300 and hook portions 110 of proximal portion 104 of distal element 100 together form a fastener pair. Proximal face 322 of proximal element 300 has formed therein recessed features 310. Proximal element 300 has a distal facing surface 330. Referring to FIGS. 3A through 3C which depict distal element 100, axial portion 116, transverse portion 118, and distally facing portion 120 together make up hook portions 110 of distal element 100. Transverse portions 118 have formed thereon beveled surfaces 122. As best seen in FIGS. 4A through 4C, recessed features 310 of proximal element 300 have a medially extending portion 312, proximally extending portion 314, and distal-medial facing beveled surface 316.

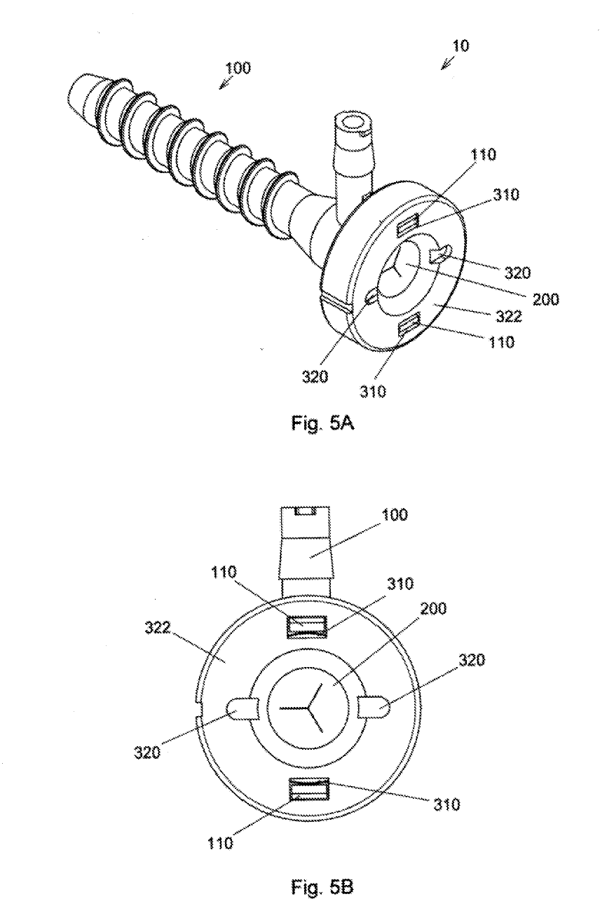

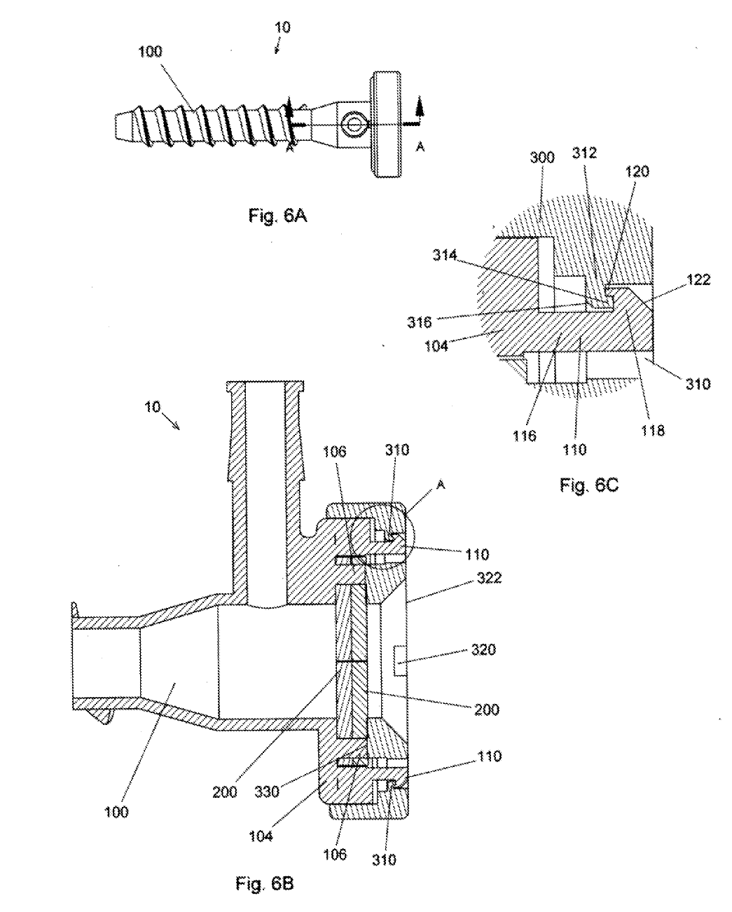

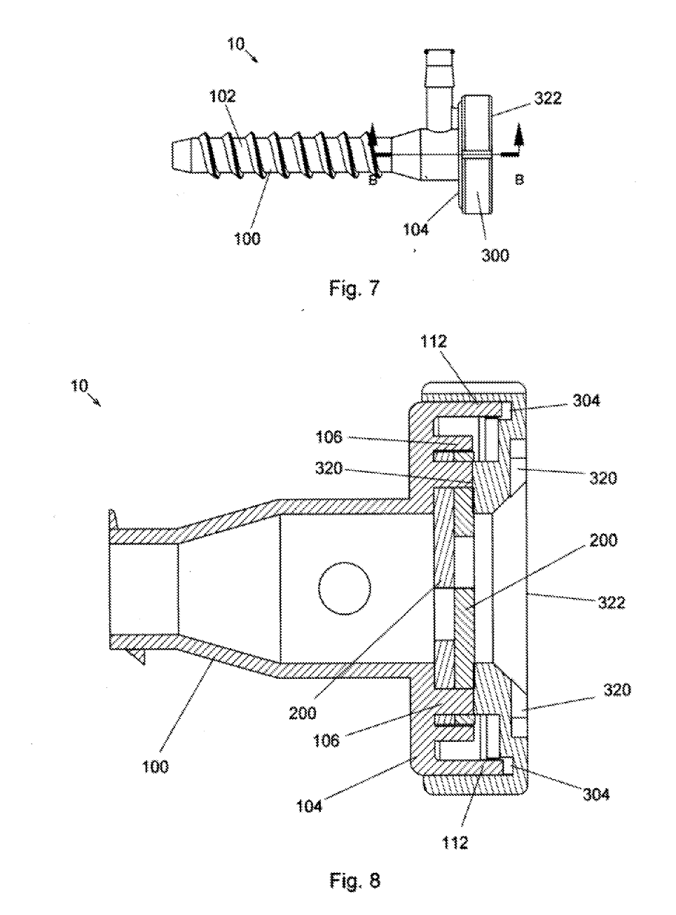

FIGS. 5 through 8 depict cannula 10 fully assembled with proximal portion 300 irremovably mounted to distal element 100. FIGS. 6A through 6C depict the mechanical interlocking of hook portions 110 of distal element 100 with recessed features 310 to prevent axial movement in the proximal direction of proximal element 300. Assembly is accomplished in the following manner. Seal elements 200 are positioned in proximal portion 104 of distal element 100. Proximal element 300 is aligned with distal element 200, hook portion 110 of distal element being partially inserted into the openings of recessed features 310. Axial force is applied to proximal element 300 so as to compress seals 200 and flex proximal portion 300. Beveled surfaces 122 of hook portions 110 acting with beveled surfaces 316 of recessed portions 310 cause hook portions 110 to flex inward, the flexure increasing with increasing axial movement of proximal element 300 relative to axial element 100. When proximal element 300 has been sufficiently advanced axially relative to distal portion 100, portions 118 and 120 of hook portions 110 protrude proximally beyond portions 312 and 314 of recessed portions 310 such that hook portions 110 can return to their free-state (un-deflected) condition. With the hook portions in their free-state position, portions 118 and 120 of hook portions 110 and portions 312 and 314 of recessed portions 310 interlock in a manner that prevents proximal movement of proximal portion 300 relative to distal portion 100. Additionally, portions 314 of recessed portions 310 in cooperation with portions 120 of hook portions 110 prevent deflection of hook portions 110 as would be required for disassembly of proximal element 300 from distal element 100. As best seen in FIG. 8, distal facing surface 330 of element 300 is in contact with the proximal ends of pins 106 of distal element 100 thereby prevent distal axial movement of element 300. Alignment protrusions 112 of distal element 100 and slots 304 of proximal element 300 maintain angular alignment between elements 100 and 300.

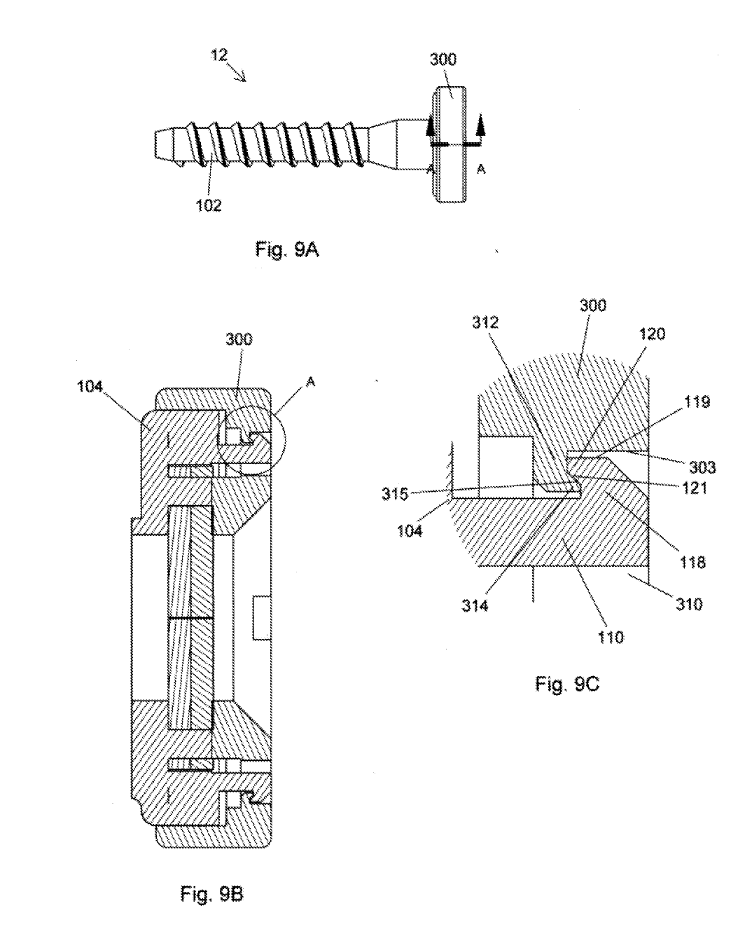

An alternate embodiment that may be optionally disassembled after assembly (that is, wherein proximal element 300 may be demounted from distal element 100 after assembly) is depicted in FIGS. 9A through 9C. Except as specifically indicated, in all aspects cannula 12 is identical to cannula 10. Portions 314 of recessed portions 310 and portions 120 of hook portions 110 have formed on them complimentary beveled surfaces 315 and 121 respectively such that by placing a blade-like device into the gaps between surfaces 119 of portions 118 of hook portions 110 and surfaces 303 of proximal element 300 and imparting a separating force between the surfaces, hook portions 110 may be deflected such that proximal element 300 is released from distal element 100. Unlike cannula 10, which is intended to be a single-use device, cannula 12 is intended as a reusable device. As such, it may be disassembled, with distal element 100 and proximal element 300 optionally formed from a more durable polymeric material such that following one or more uses, sealing elements 200 may be replaced and additional uses of cannula 12 realized.

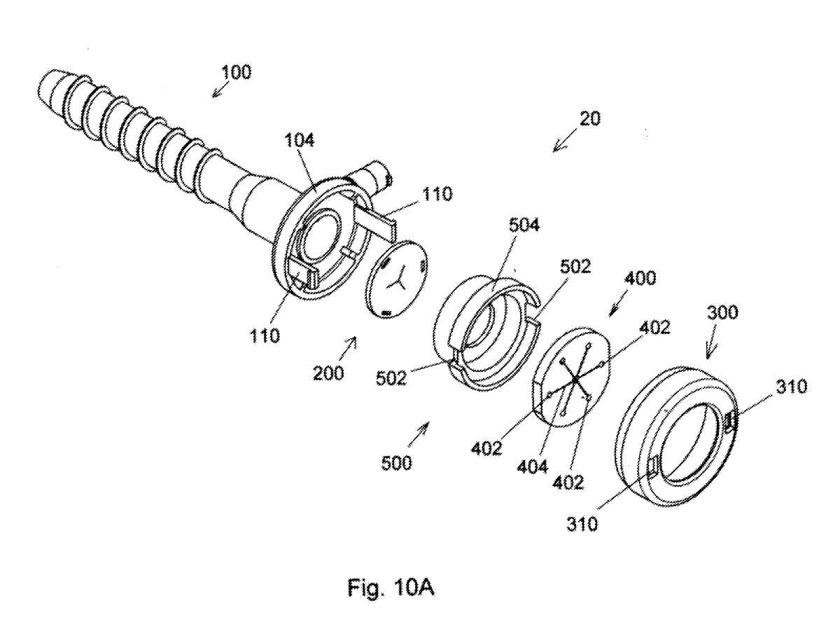

When a suture passing from a cannula is placed under tension, the seal is often deformed and pressurized fluid from within the joint sprays from the proximal end of the cannula. Frequently the stream of fluid will strike the surgeon. Referring now to FIGS. 10A and 10B, alternate embodiment cannula 20 formed in accordance with the principles of this invention has a spray shield 400 to prevent streams of fluid which escape the seal 200 from spraying at the surgeon. Spray shield 400, formed from a suitable elastomeric material, has radial slits 404 terminating in holes 402 so as to form spray-deflecting flaps between the slots, and holes through which fluid leaking from seal 200 may flow as a low-velocity stream. Spray shield 400 and seal 200 are positioned within mid-element 500. The assembly of seal 200, mid-element 500 and spray shield 400 is then positioned in the proximal end 104 of distal element 100 and proximal element 300 is mounted to element 100 in the same manner as for cannulae 10 and 12.

The joining of plastic components may also be reliably accomplished by heat-staking, a process in which one or more features of one of the components of the final assembly is thermally deformed so as to create a mechanical barrier to disassembly. For instance, an assembly may have mating features on its component elements such that, when assembled, a protuberance of a first element is positioned within an opening of a second element, the distal end of the protuberance extending beyond a surface of the second element. The protruding distal end of the protuberance is thermally deformed so as to locally increase its size so as to prevent retraction through the mating opening. Heat-staking is a reliable method for securing assemblies since the strength of an individual heat-staked element is determined by the dimensions of the deformed region and the shear strength of the polymeric material. Also, heat-staked components may be visually inspected to verify their integrity, a feature lacking on bonds formed by ultrasonic welding or other means.

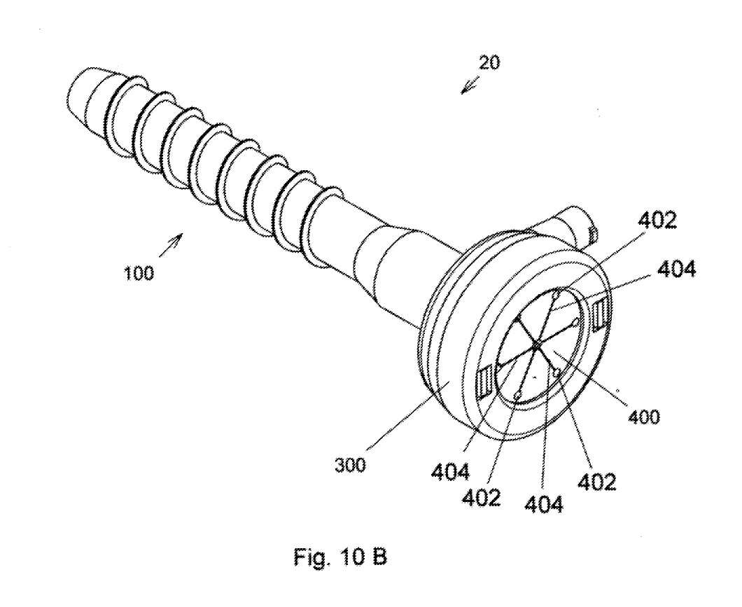

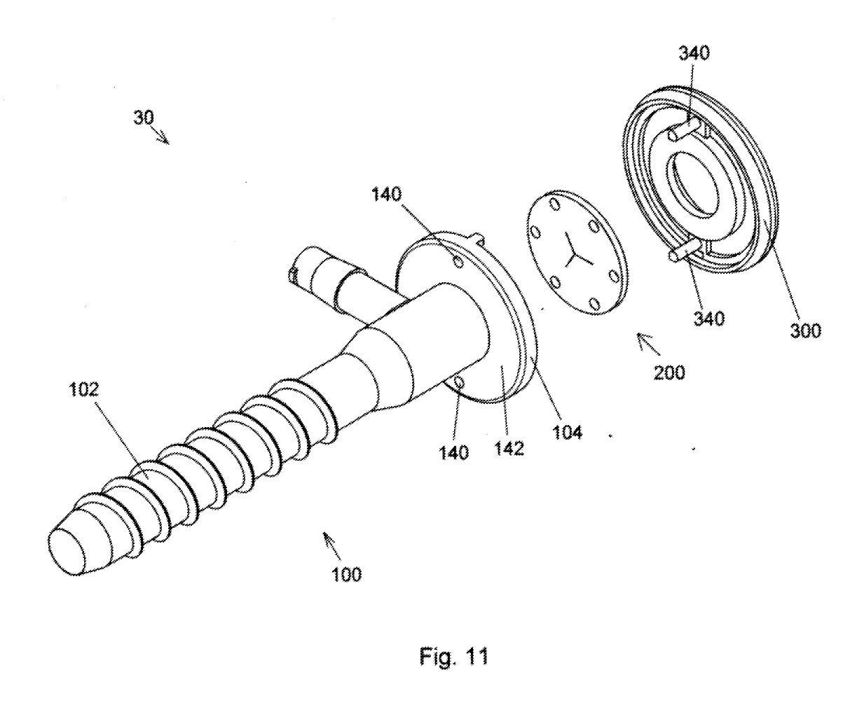

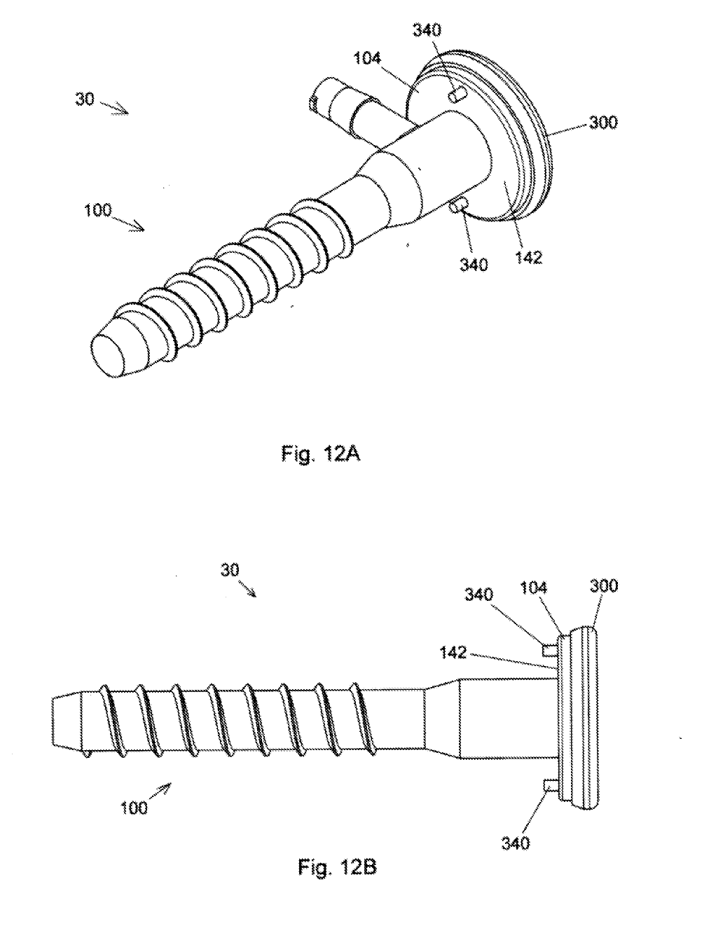

FIG. 11 depicts the components for an alternate embodiment cannula 30 formed in accordance with the principles of this invention and arranged for assembly. Proximal element 300 has formed thereon distally extending portions 340. Distal element 100 has formed in the distal facing surface 142 of its proximal portion 104 holes 140 which are sized and positioned to receive portions 340 upon assembly. FIGS. 12A and 12B depict the elements of cannula 30 assembled with the distal portions of distally extending portions 340 protruding beyond surface 142 of proximal portion 104 of cannula distal portion 100. FIGS. 13A through 13C depict cannula 30 after final assembly. As best seen in FIG. 13B, the portions of portions 340 extending beyond surface 142 of distal element 100 have been thermally deformed (heat-staked) to a hemispherical shape having a proximal diameter larger than that of holes 140 of distal element 100. This deformation prevents withdrawal of portions 340 from holes 140 and thereby preventing disassembly of cannula 30.

Cannula 20 of FIGS. 10A and 10B, with its integral spray shield 400, requires a distal element 100 and proximal element 300 formed in a manner which allows the assembly therebetween of mid-element 500 with sealing element 200 and spray shield 400. This construction requires the construction of the molds configured to produce not only the distal element 100 and proximal element 300, but also the mid-element 500. Alternate embodiment cannulae with integral spray shields are anticipated in which cannulae 10 or 12 as previously described herein are modified through the additional of a proximally mounted spray suppression assembly. The benefits to be realized from this construction are reduced tooling and inventory costs since “standard” cannulae (that is, without spray shields) may be modified to produce cannulae with spray shields. Additionally, the tooling and manufacturing costs for the spray suppression assemblies are low since the configuration of the elements of the assemblies are designed for low-cost tooling and manufacturing. That is, while the elastomeric spray shield must be produced in its own injection mold, the two other rigid components may be molded in what is commonly called a “family mold”, that is, a single mold in which multiple related parts are formed simultaneously with each cycle of the molding machine.

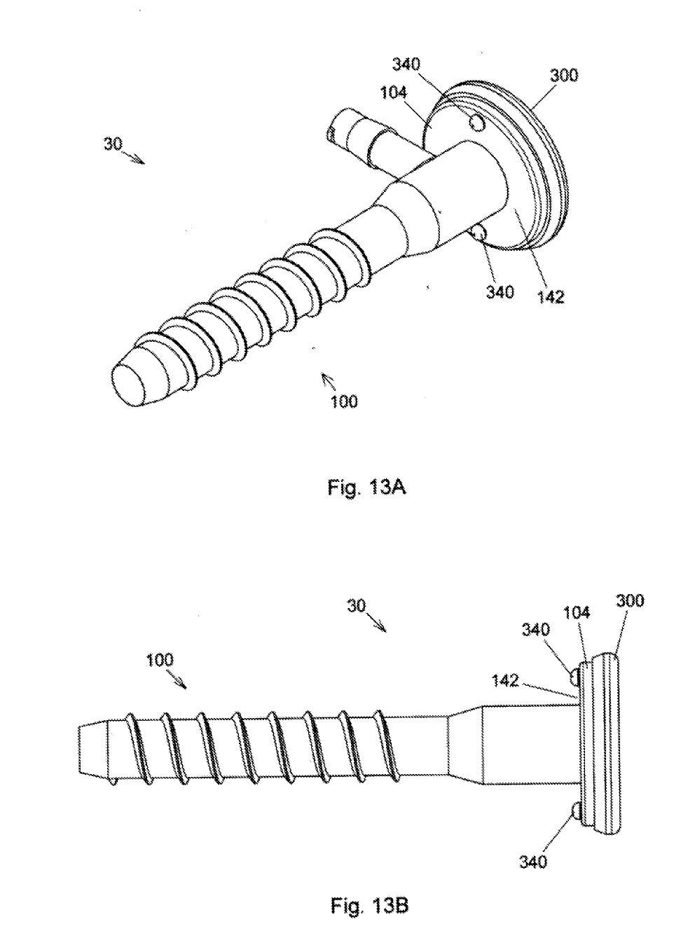

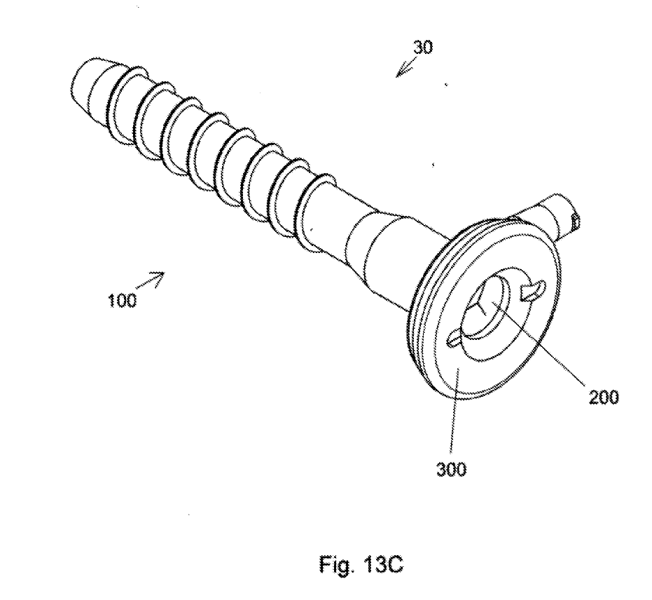

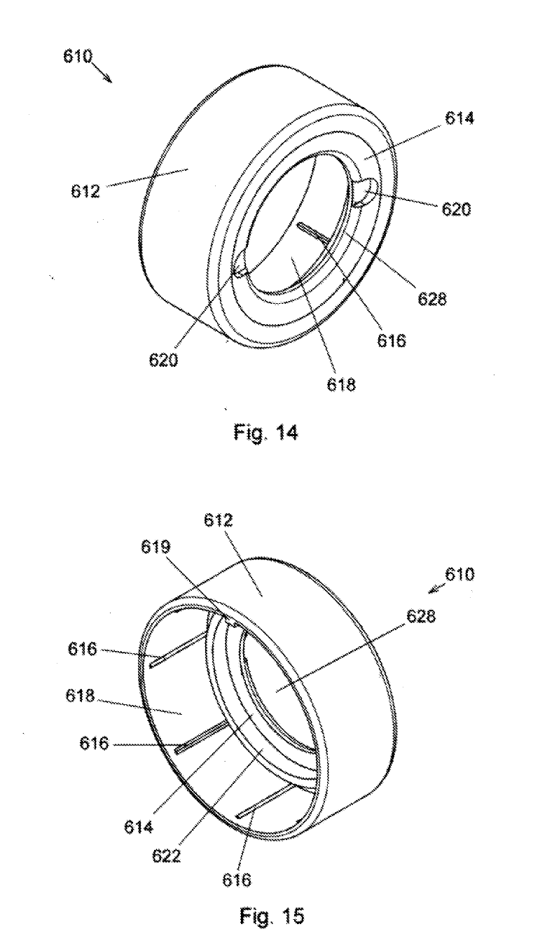

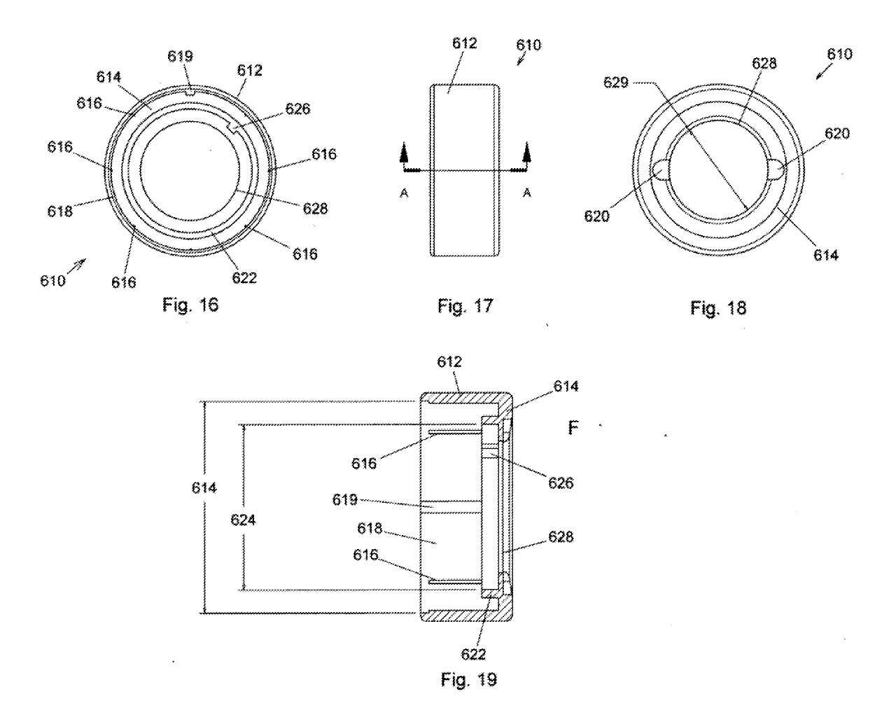

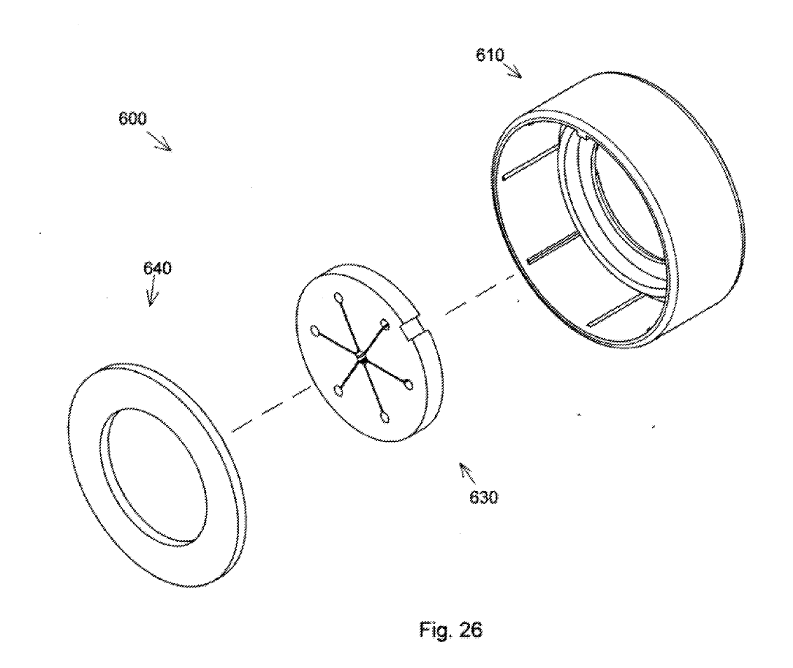

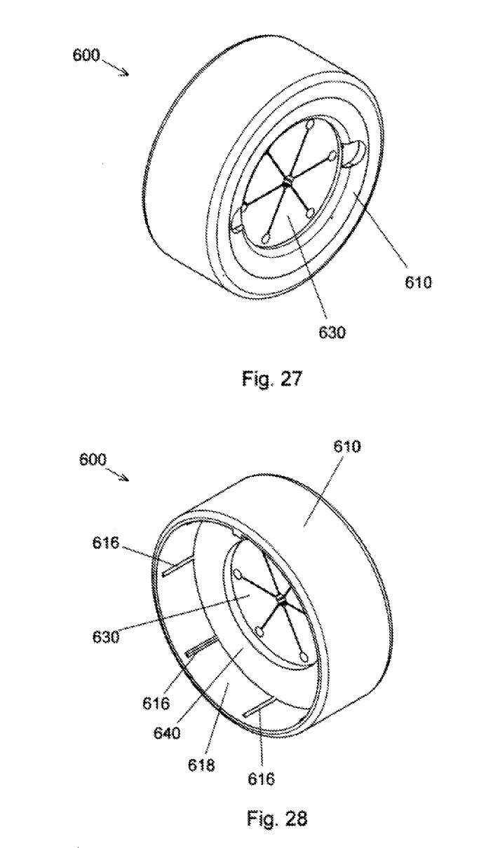

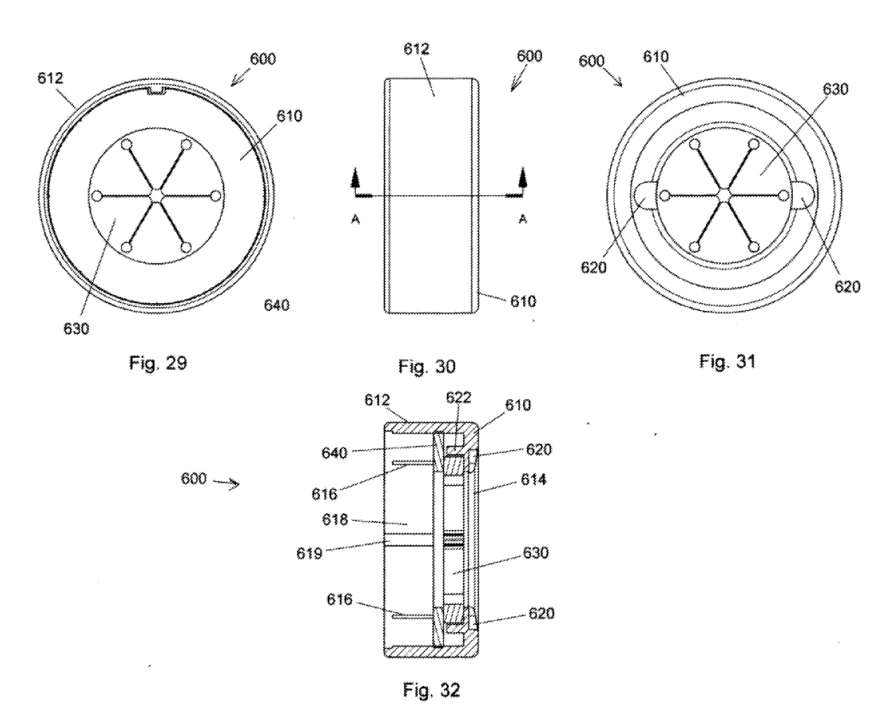

FIGS. 14 through 19 depict tubular body element 610 for a simplified spray suppression assembly 600 (FIGS. 26 through 32) which may be assembled to cannula 10 (FIGS. 1 through 8). Body element 610 has a tubular distal portion 612 having an inner diameter 614 sized to allow mounting of body element 610 to proximal element 300 of cannula 10, and inwardly extending axial ridges 616 (commonly called “crush ribs”) on inner cylindrical surface 618 along with alignment key 619. Proximal inwardly extending flange 614 of body element 610 has formed in its proximal face slots 620 having the form and function of slots 320 of proximal element 300 of cannula 10. Flange 614 has formed on its distal surface flange 622 which forms a cylindrical pocket of diameter 624 that has formed therein alignment key 626. Flange 614 defines a circular opening 628 of diameter 629. Body 610 is formed of a suitable rigid polymeric material.

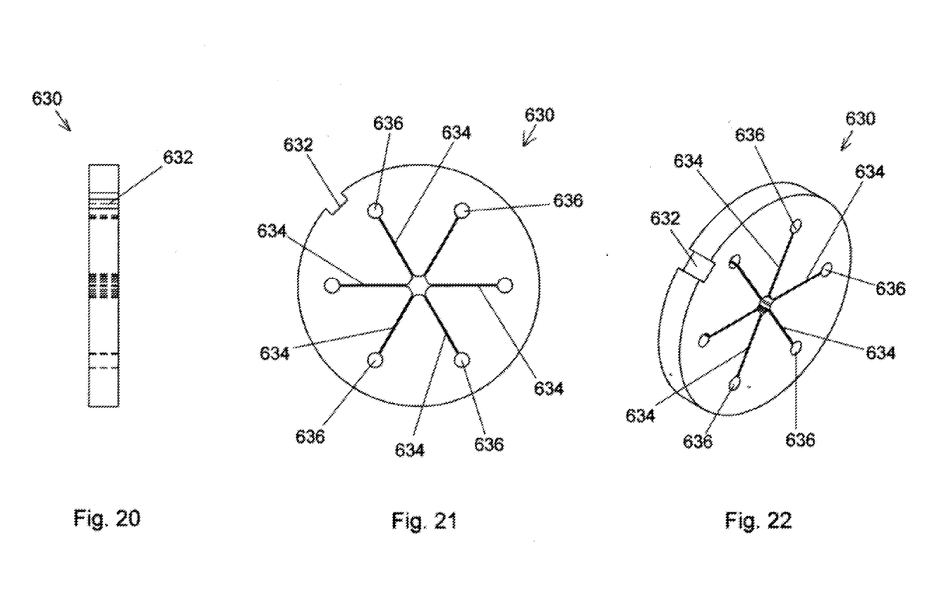

FIGS. 20 through 22 depict a flexible polymeric spray shield 630 having a diameter and thickness selected to allow the placement of shield 630 in the cylindrical pocket formed by flange 614 of body element 610, angular alignment of spray shield 630 to body 610 being established by alignment notch 632 of shield 630 and alignment key 626 of body 610. Shield 630 has formed therein a pattern of radially extending slots 634 terminating in holes 636 so as to form therebetween deformable flaps 636.

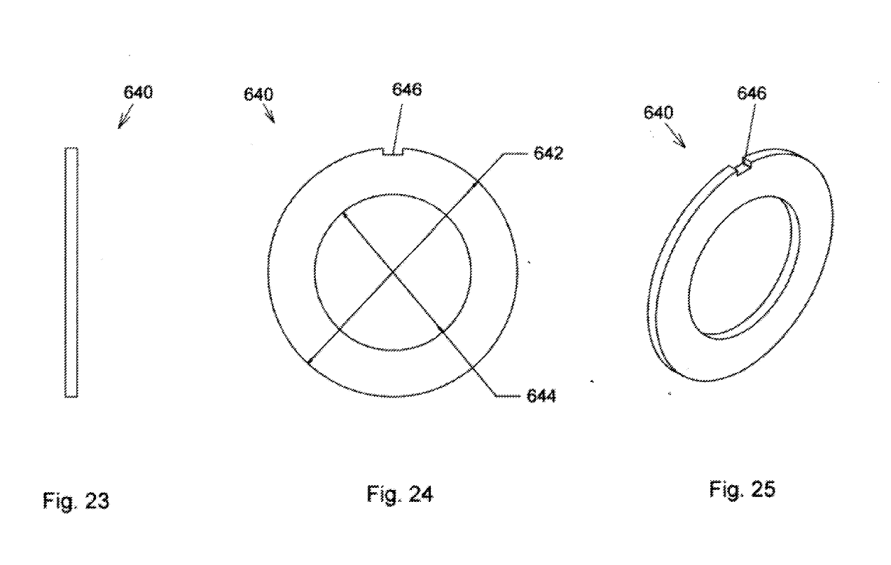

FIGS. 23 through 25 depict a retaining ring 640 formed of a suitable rigid polymeric material having an outer diameter 642 slightly less than diameter 614 of distal portion 612 of body 610 Inner diameter 644 is approximately equal to diameter 629 of circular opening 628 of flange 614 of body 610.

As seen in FIGS. 26 through 32 depicting spray suppression assembly 600, spray shield 630 is positioned in the circular recess created by flange 622 of proximal flange 614 of body 610, and is retained in that position by retaining ring 640 positioned within tubular portion 612 of body 610. Retaining ring 640 has a diameter which causes interference between protruding axial ridges 616 of inner surface 618 of body 610 so as to prevent retaining ring 640 and spray shield 630 from being dislodged from body 610.

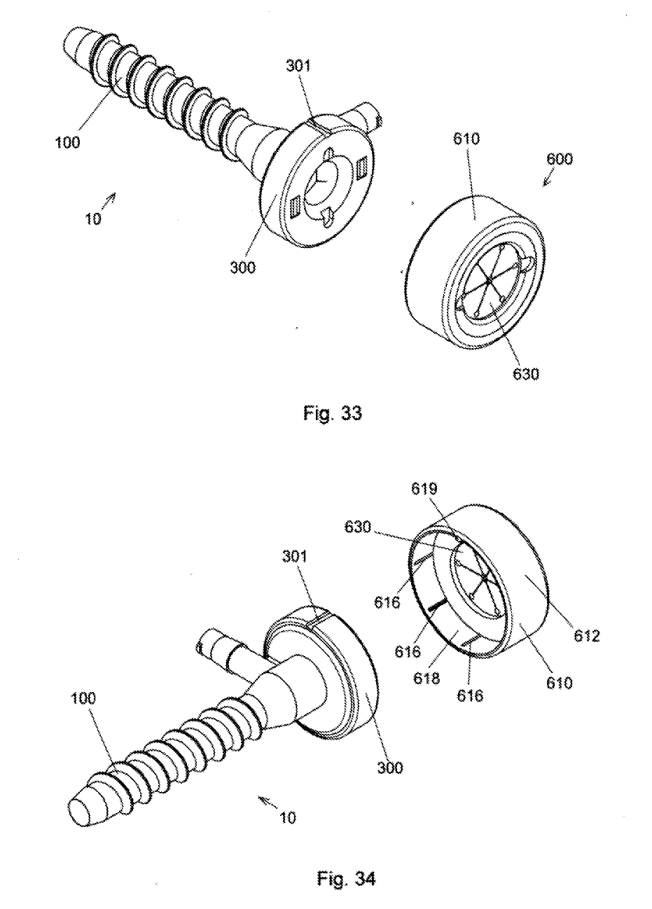

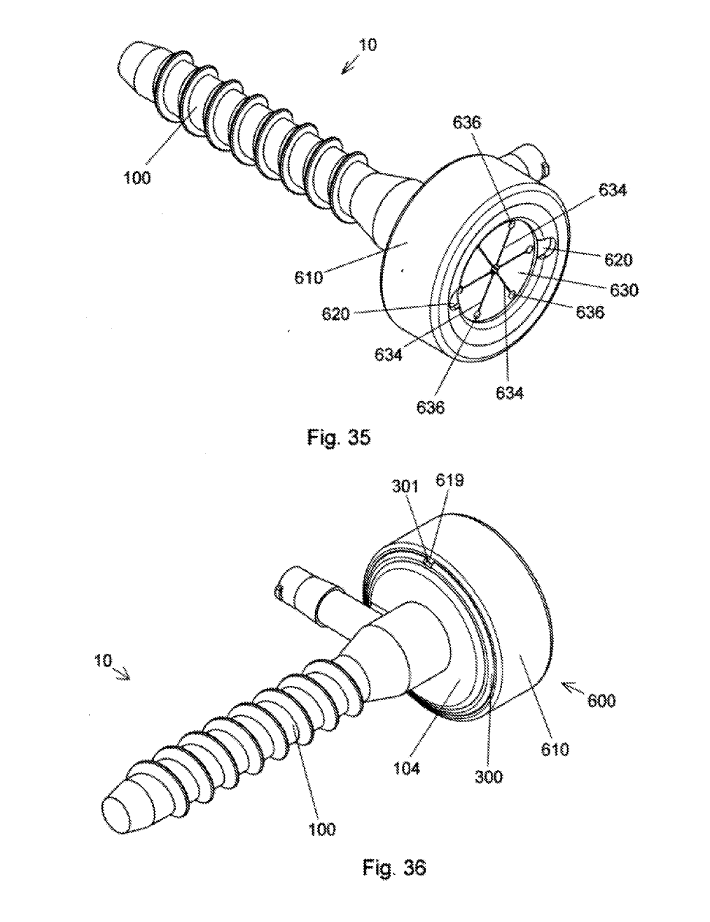

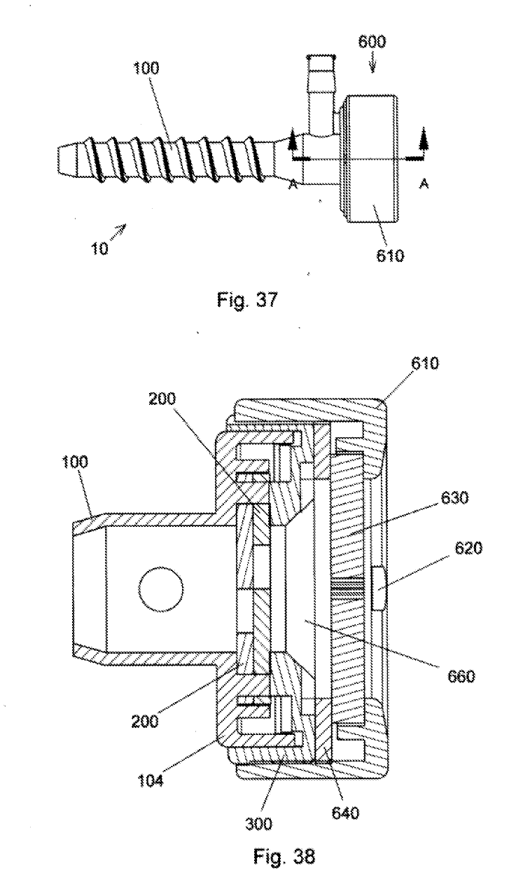

FIGS. 33 and 34 depict cannula 10 (FIGS. 2 through 9) and spray suppression assembly 600 positioned for assembly wherein spray suppression assembly 600 is mounted to proximal element 300 of cannula 10, interference between crush ribs 616 and the outer cylindrical surface of element 300 preventing demounting. Alignment key 619 of element 610 of assembly 600 and axial slot 301 of proximal portion 300 provide angular alignment between spray suppression assembly 600 and cannula 10. As with previous embodiments, no solvent bonding or ultrasonic welding is used. As seen in FIG. 38, spray shield 630 is proximally displaced from seals 200 so as to create therebetween void 660. Fluid leaking past sealing elements 200 fills void 600 thereby converting high velocity flow past sealing elements 200 into low velocity flow which escapes through the flaps formed by slits 634 and holes 636 thereby preventing spraying of fluids on the surgeon and surrounding area.

Spray suppression assembly 600 relies on interference between crush ribs 616 of body 610 and retaining ring 640 and between crush ribs 616 and proximal element 300 to irremovably mount the elements one to another. In an alternate embodiment of the instant invention, crush ribs 616 are eliminated and irremovable assembly of the elements is accomplished by an interference fit between the respective cylindrical surfaces. In yet another embodiment, the spray suppression assembly may be removable from the cannula.

In yet another alternate embodiment, the principles of the instant invention are applied to a cannula having a flexible distal portion able to accommodate curved instruments and those having irregularly shaped distal portions that will not fit through the lumen of a conventional rigid cannula. In the flexible cannulae of the instant invention, the rigid distal portion 100 of previous embodiments is replaced by an assembly having a rigid proximal portion and a flexible distal portion, the flexible distal portion being affixed to the rigid proximal portion without the use of bonding agents, but rather through a unique configuration of complementary features and a retaining collar.

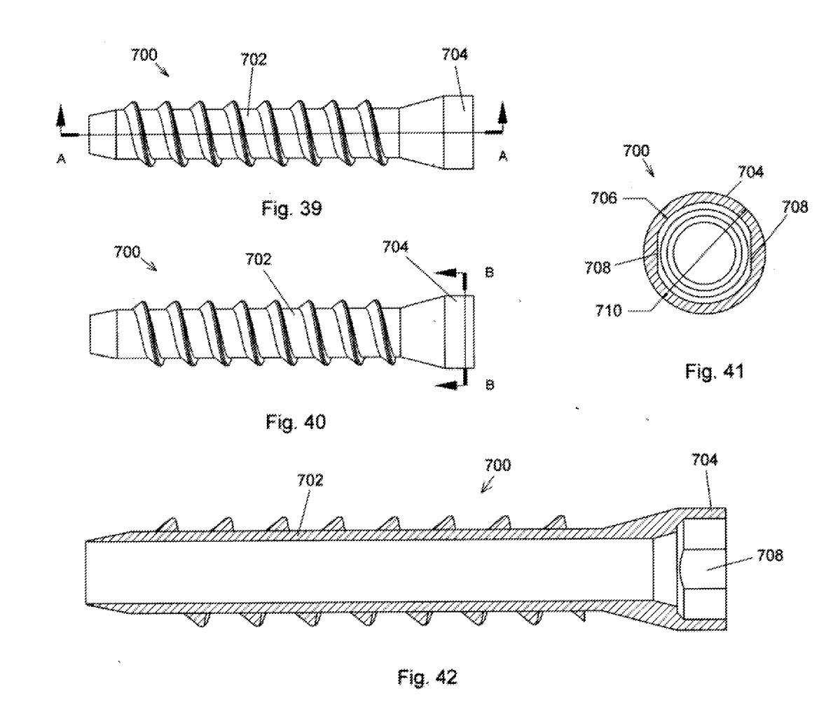

The elastomeric distal portion 700 for a cannula with a flexible distal portion according to the instant invention is depicted in FIGS. 39 through 42. Distal element 700 has an elongate distal tubular portion 702 which may optionally be threaded, and a proximal tubular portion 704 of outer diameter 710, radial surfaces of radius 706 and laterally opposed flats 708.

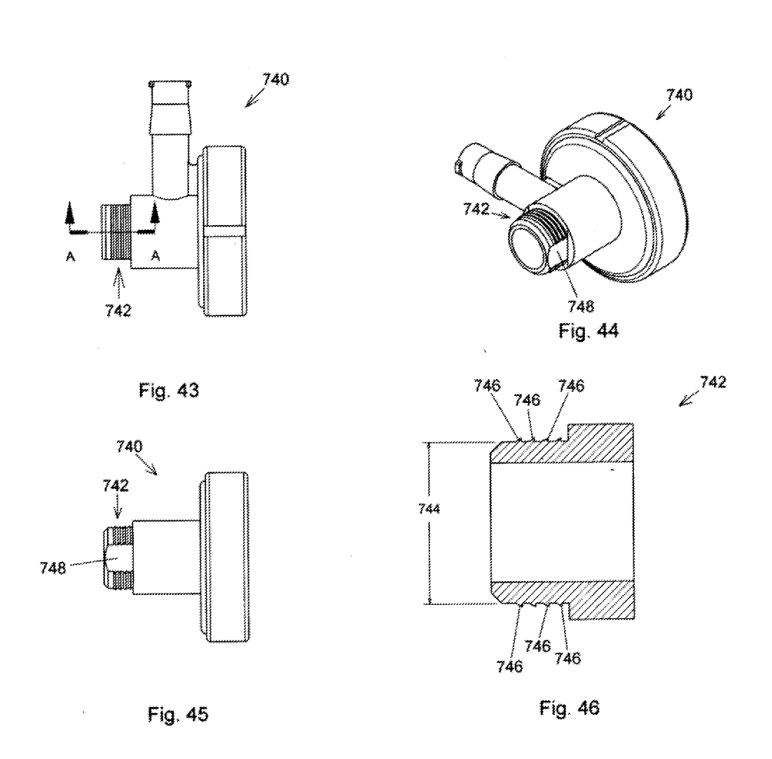

The proximal assembly 740 for a cannula with a flexible distal portion according to the instant invention is depicted in FIGS. 43 through 46. Proximal assembly 740 is identical in form and function to cannula 10 except as described hereafter. Distal portion 742 of assembly 740 has formed on its distal end tubular portion 742 of outer diameter 744 with wedge-shaped ridges 746 formed on its outer radial surfaces, and laterally opposed flats 748 formed thereon. The form of distal portion 742 of proximal assembly 740 is complementary to the form of proximal portion 704 of distal element 700. Diameter 744 of distal portion 742 of proximal assembly 740 may be greater than the sum of radii 706 of proximal portion 704 of elastomeric distal element 700 so that when elastomeric distal element 700 is mounted to distal portion 742 of proximal assembly 740 proximal portion 704 is stretched and wedge-shaped ridges 746 penetrate the inner radial surfaces of proximal portion 704 of elastomeric distal element 700.

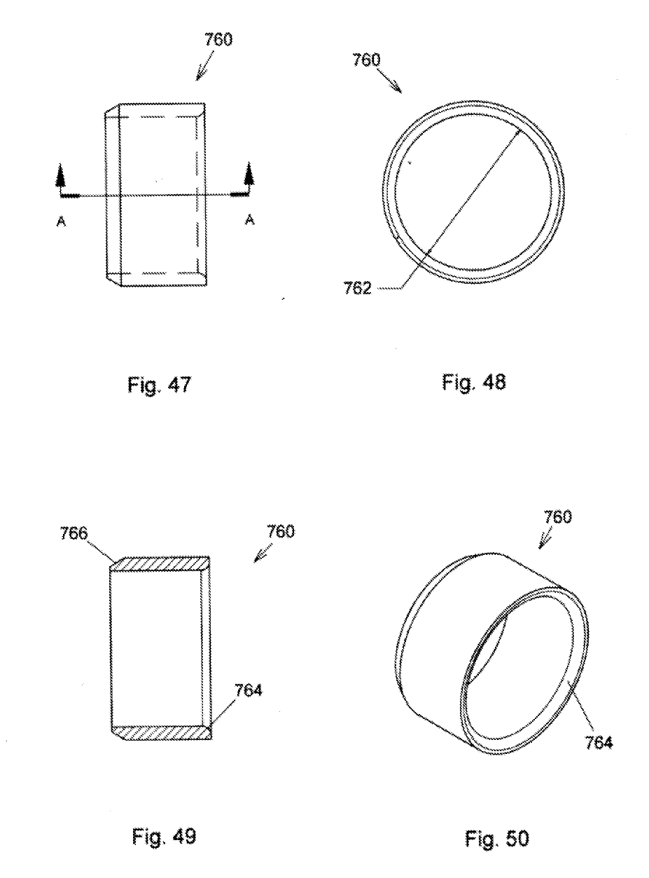

FIGS. 47 through 50 depict a tubular collar 760 having an inner diameter 762 approximately equal to outer diameter 710 of proximal portion 704 of elastomeric element 700, a radiused inner proximal edge 764 and a chamfered distal outer edge 766.

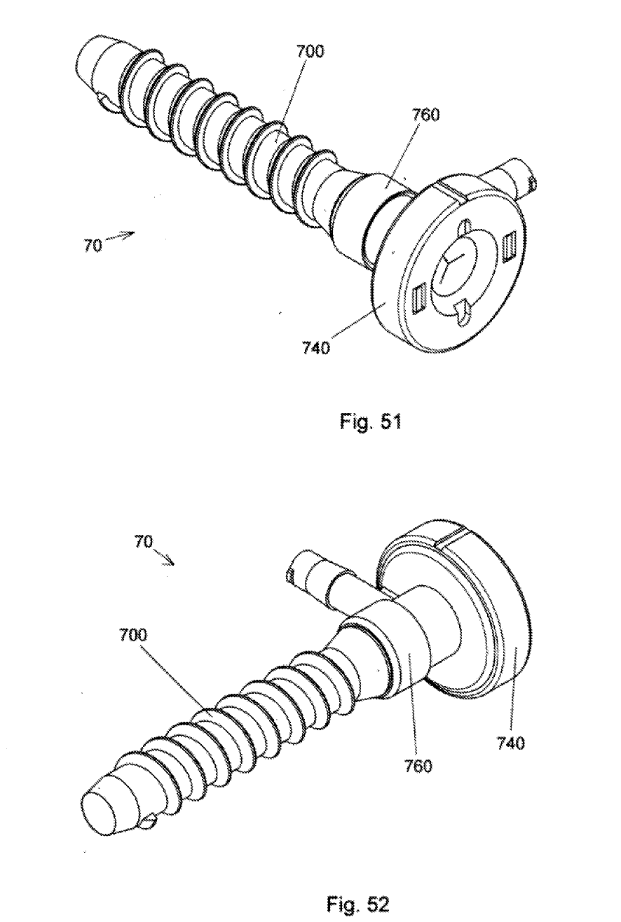

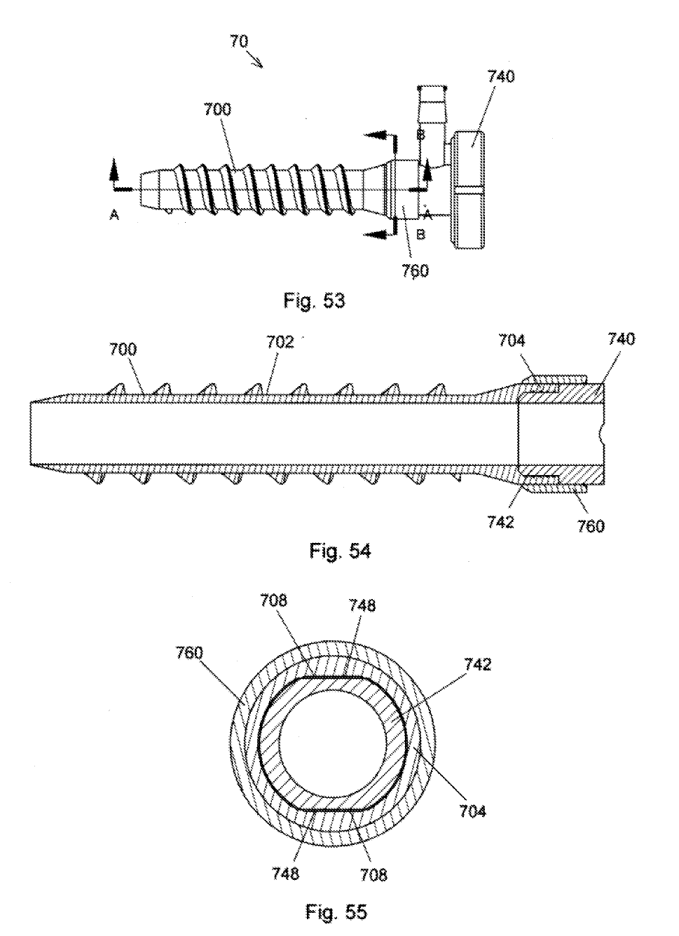

Cannula 70 having a flexible distal portion and formed in accordance with the principles of this invention is depicted in FIGS. 51 through 55. Elastomeric distal portion 700 is mounted to proximal assembly 740 as previously described. Collar 760 is positioned about proximal portion 704 of elastomeric distal element 700 so as to place portion 704 under compression and prevent demounting of element 700 from proximal assembly 740. Collar 760 may be made from either a suitable polymeric or a suitable metallic material. In a preferred embodiment collar 760 is inelastically deformed after positioning on the assembly to produce increased compressive pressure on the assembly.

### INDUSTRIAL APPLICABILITY

As noted previously, the present invention is directed to a simplified, low cost arthroscopic sealing cannula having improved efficiency and reduced manufacturing costs. In particular, by replacing the conventional thermal and chemical bonding means with a mechanical joining system, the present invention provides for a substantial reduction in manufacturing costs, a dramatically simplified validation process as well as a reduced opportunity for failure. Cannulae formed in accordance with the principles of this invention may be assembled using integral fastener pairs formed with hooked sections, using heat-staked elements, or using pressed together elements that have interfering and/or friction fit features. The cannulae may optionally have a spray shield or may have a flexible distal element. The choice of the assembly method for a given device and combinations and variations of placement of these methods fall within the scope of this invention.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

The invention has been illustrated by reference to specific examples and preferred embodiments. However, it should be understood that the invention is intended not to be limited by the foregoing description, but to be defined by the appended claims and their equivalents.

What is claimed:

1. A cannula assembly comprising:
a. a proximal hub element comprising (i) a central opening configured to receive surgical instruments, (ii) a planar annular body portion that includes a first component of a mating fastener pair, and (iii) a distally projecting flange portion;
b. a distal tubular element comprising (i) an elongate tubular distal portion, (ii) a flared proximal portion that includes a second component of said mating fastener pair, and (iii) a proximally facing raised rim extending from said flared proximal portion, and
c. one or more sealing membranes,
wherein components (a)-(c) are assembled together such that said first and second components of said mating fastener pair mechanically interlock so as to securely fasten said proximal hub element to said distal tubular element and prevent relative movement and/or disengagement thereof.

2. The cannula assembly of claim 1, wherein said first and second components of said mating fastener pair together comprise a plurality of integral projecting hooks that mate with a corresponding plurality of integral recessed features.

3. The cannula assembly of claim 2, wherein:
a. each of said plurality of integral projecting hooks comprises an axial portion, a transverse portion, a beveled surface, and a distal tip; and
b. each of said plurality of integral recessed features comprises a medially extending portion, a distal facing beveled surface, and a proximally projecting tip portion;
c. wherein the mechanical interlocking of said hooks and recessed features arises from the engagement of said respective beveled portions.

4. The cannula assembly of claim 2, wherein said plurality of integral projecting hooks proximally project from the flared proximal portion of said distal tubular element and said plurality of integral recessed features are disposed in the planar annular body portion of said proximal hub element.

5. The cannula assembly of claim 2, wherein said plurality of integral projecting hooks distally project from the planar annular body portion of said proximal hub element and said plurality of integral recessed features are disposed in flared proximal portion of said distal tubular element.

6. The cannula assembly of claim 1, wherein said proximal hub element may be repeatedly disassembled from said distal tubular element.

7. The cannula assembly of claim 1, wherein the proximal hub element is permanently affixed to said distal tubular element.

8. The cannula assembly of claim 2, wherein said recessed features comprise holes through which said plurality of hooks extend, further wherein an exposed portion of said plurality of hooks is thermally deformed to prevent withdrawal of said hooks from said holes and thereby ensure permanent affixation between said proximal hub element and said distal tubular element.

9. The cannula assembly of claim 1, wherein said flared proximal portion of said distal tubular element retains said one or more sealing membranes.

10. The cannula assembly of claim 9, wherein said flared proximal portion is provided with one ore more integral pins that engage with mating holes provided on said one or more sealing membranes.

11. The cannula assembly of claim 1, wherein:
a. said planar annular body portion includes one or more integrated slots; and
b. said proximally facing raised rim includes one or more proximally facing alignment protrusions disposed about its periphery;
c. wherein said one or more alignment protrusions cooperatively engage with said one of more slots so as to establish and maintain proper angular alignment between said proximal element and said distal element.

12. The cannula assembly of claim 1, further comprising (d) an elastomeric spray shield and (e) an annular retaining body.

13. The cannula assembly of claim 12, wherein said elastomeric spray shield has a plurality of radial slits terminating in holes that together form spray-deflecting flaps between the slots.

14. The cannula assembly of claim 13, wherein said annular retaining body comprises a proximal facing surface having a first raised rim at the periphery configured to retain said spray shield and a distal facing surface comprising a second raised rim at the periphery configured to retain said one or more sealing gaskets, further wherein said elastomeric spray shield, annular retaining body, and said one or more sealing gaskets are assembled together and disposed between said proximal and distal elements.

15. The cannula assembly of claim 13, wherein said elastomeric spray shield and annular retaining body are assembled together and that assembly is then mounted to the proximal hub element of said cannula assembly.

16. The cannula assembly of claim 15, wherein said retaining body has an inner cylindrical surface provide at least one alignment key that mates with a corresponding number of axial slots disposed on said proximally facing raised rim of said proximal hub element.

17. The cannula assembly of claim 16, wherein said inner cylindrical surface of said retaining body is further provided with one or more inwardly extending axial ridges that give rise to an irremovable interference fit between said retaining body and said proximal hub element.

18. The cannula assembly of claim 1, wherein said proximal hub element and said distal tubular element are each integrally molded from a rigid polymeric material.

19. The cannula assembly of claim 1, wherein said elongate tubular distal portion of said distal element is fabricated from an elastomeric material while said flared proximal portion and said proximally facing raised rim are fabricated from a rigid material.

20. The cannula assembly of claim 1, wherein said tubular distal element further comprises external threads.
